(19) United States
(12) Patent Application Publication
(10) Pub. No.: US 2019/0198171 A1
(43) Pub. Date: Jun. 27, 2019

(54) INTERACTIVE PHYSIOLOGY MONITORING AND SHARING SYSTEM

(71) Applicant: Linctronix Ltd., New Taipei City (TW)

(72) Inventors: Yu-Shun LIN, New Taipei City (TW); Kuei-Han CHEN, New Taipei City (TW)

(21) Appl. No.: 16/230,490

(22) Filed: Dec. 21, 2018

(30) Foreign Application Priority Data
Dec. 27, 2017 (TW) 106145920

(51) Int. Cl. G16H 50/20; G16H 10/60

(52) U.S. Cl. CPC G16H 50/20; H04W 4/80; G16H 10/60

(57) ABSTRACT

The present invention provides an interactive physiology monitoring and sharing system to be used in a social media. The present invention comprises a Bluetooth physiological sensor and a server. The Bluetooth physiological sensor is used for sensing and sending a physiological data. The server is connected to the social media and the Bluetooth physiological sensor for receiving the physiological data. An agent is pre-stored in the server. Wherein, when the server receives a user message from the social media, the agent analyzes the user message and the physiological data to generate and send a feedback message to the social media. Through the social media and the agent, users can easily share their health status and receive health advice without wasting medical resources.

### CROSS-REFERENCE TO RELATED APPLICATIONS

This application claims the priority benefit of Taiwan Application Serial No. 106145920 filed Dec. 27, 2017 the disclosure of which is incorporated herein by reference.

### BACKGROUND OF THE INVENTION

### Field of the Invention

The present invention provides an interactive physiology monitoring and sharing system, more particularly, to a system sharing the physiology information to the social media via Bluetooth and internet, and then making health advice by the interaction between the social media and the authorized terminal.

### Description of the Prior Art

The medical and health care system model supported by the internet technology is a new trend in the digital health today. The digital health refers to a variety of medical applications of communication technology, which not only includes the informatization of the internal medical process in the hospital, but also includes the regionalization of the medical information sharing and health service. The former is the foundation of digital health, and the latter is the advanced form of the digital health. In the remote monitoring of the regional health services, the patient with chronic diseases does not have to go to the hospital since the home physiology detector can transmit the monitored data to the medical professional. Then, the patient can obtain healthy advice and recommendation after the medical professional reviews the data.

In the prior art, the physiological data detected by the physiological detector is uploaded and stored in the cloud server. The medical professional installs a specific program in a terminal device to obtain the physiological data by accessing the server. Then, based on the physiological data, the medical professional puts forward health advice to the examiner and the parties who is concerned.

However, the medical professional cannot be on call throughout the day. In addition, since the physiological data detected by the physiology detector usually can be analyzed and interpreted by the computer system program, to consult directly with medical professional becomes a waste of medical resources. Moreover, in recent years users have been tired of being asked to download different applications one by one. Therefore, the above situation becomes a problem to be solved in the digital health treatment.

### SUMMARY OF THE INVENTION

In response to the above-mentioned problems, an objective of the present invention is to provide an interactive physiology monitoring and sharing system. The physiological data is uploaded and stored in the server. The server then shares the physiological information to the social media now in common use. Moreover, the user can interact with the server by social media to make an easy and convenient healthy consultation.

An interactive physiology monitoring and sharing system of the present invention is applied to a social media, and the system comprises a Bluetooth physiological sensor and a server. The Bluetooth physiological sensor is used for sensing and sending physiological data. The server is connected to the social media and the Bluetooth physiological sensor for receiving the physiological data, wherein an agent is pre-stored in the server. When the server receives a user message from the social media, the agent analyzes the user message and the physiological data for generating and sending a feedback message to the social media.

In an embodiment, when the server receives the physiological data, the agent generates and sends a report message containing the physiological data to the social media.

In an embodiment, the interactive physiology monitoring and sharing system further comprises an authorized terminal connected to the social media. Wherein, the authorized terminal is used for sending the user message to the social media, and receiving the report message and the feedback message from the social media.

In an embodiment, the interactive physiology monitoring and sharing system further comprises a plurality of authorized terminals. Wherein, when the Bluetooth physiological sensor sends the physiological data to the server, the server sends the report message to the authorized terminals respectively via the social media.

In an embodiment, when a first authorized terminal sends the user message to the server, the server sends the feedback message to the authorized terminals respectively via the social media.

In an embodiment, the interactive physiology monitoring and sharing system further comprises a Bluetooth gateway. Wherein the Bluetooth gateway is connected to the Bluetooth physiological sensor through Bluetooth technology for receiving a Bluetooth message containing the physiological data, and the Bluetooth gateway is connected to the server via the TCP/IP or other protocols for sending a sensing message containing the physiological data to the server.

In an embodiment, the interactive physiology monitoring and sharing system further comprises a plurality of authorized terminals. Wherein, each of the authorized terminals is used for sending the user message to the social media and receiving the report message and the feedback message from the social media. The Bluetooth message and the sensing message contain a device identifier of the Bluetooth physiological sensor respectively. The device identifier is used for corresponding to a first authorized terminal. The agent sends the report message and the feedback message to the first authorized terminal via the social media according to the device identifier.

In an embodiment, each of the authorized terminals contains a user identifier; the device identifier is used for corresponding to a first user identifier. According to the device identifier, the agent sends the report message and the feedback message to at least one authorized terminal containing the first user identifier corresponding to the device identifier.

In an embodiment, the agent generates a tag of the physiological data according to the user message, and the tag comprises an identity of the subject, data source device and validity of the physiological data.

In an embodiment, the feedback message and the report message comprise at least one of a word, a text, an audio stream, a video message, an animation, a sign, or an image respectively.

In an embodiment, the agent comprises at least one of a robotic program, a chatbot, an artificial intelligence program, or a machine learning program.

In an embodiment, the Bluetooth is any versions of the Bluetooth communication protocol defined by the Bluetooth Special Interest Group, such as the Bluetooth 2.0 protocol series, the Bluetooth 3.0 protocol series, the Bluetooth 4.0 protocol series or the Bluetooth 5.0 protocol series Bluetooth devices.

In an embodiment, the internet is a wired connection, a wireless LAN or a mobile communication network.

In an embodiment, the wired connection is Ethernet, FTTx, Power line communications, and networks able to deliver the network packets.

The wireless LAN is any versions of the 802.11 protocol defined by Wi-Fi or IEEE, such as 802.11, IEEE 802.11a, 802.11b, 802.11g, 802.11h, 802.11n, 802.11p, 802.11ac, 802.11ah, 802.11aq, 802.11ax and so on.

The mobile communications network is LoRa, Sigfox or the network agreement of Release 4, Release 5, Release 6, Release 7, Release 8, Release 9, Release 10, Release 11, Release 12, Release 13, or Release 14 released by 3GPP, or other mobile communications network agreement approved by ITU.

In summary, since the present invention sends the physiological data to the server with Bluetooth, the Bluetooth physiological sensor does not require high-cost storage and computing ability. Instead of medical professional, the agent can analyze and explain the physiological data, so the user can instantly get the corresponding report. The social media is used to make interaction between the user and the agent to save another job of installing App. Moreover, the social media can be used to make interaction between a plurality of users and the agent to understand each other's health. Furthermore, the agent may communicate with the user in natural language due to chatbot program. Then the physiological data is tagged to identify according to the user message or the physiological characteristic. At last, since the authorized sensors, accounts, and terminals are used to identify the user, the present invention could communicate with the target user.

### BRIEF DESCRIPTION OF THE APPENDED DRAWINGS

Some of the embodiments will be described in detail, with reference to the following figures, wherein like designations denote like members, wherein:

FIG. 1 shows a schematic diagram of one embodiment of the interactive physiology monitoring and sharing system of the present invention.

FIG. 2 shows a schematic diagram of another embodiment of the interactive physiology monitoring and sharing system of the present invention.

FIG. 3 shows a schematic diagram of one embodiment of the Bluetooth gateway of the present invention.

FIG. 4 shows a schematic diagram of another embodiment of the interactive physiology monitoring and sharing system of the present invention.

FIG. 5A and FIG. 5B show a schematic diagram of an embodiment of the interaction between the authorized terminal and the server displayed in the interface of the authorized terminal respectively.

The advantages, spirits, and features of the present invention will be explained and discussed with embodiments and figures as follows.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications can be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

In the description of the present specification, the terminologies “in an embodiment”, “in another embodiment”, or “in some embodiments” means that the specific feature, structure, material or characteristic of the present embodiment is involved in at least one embodiment of the present invention. In the description of the present specification, the schematic representation of the mentioned terminologies does not necessarily refer to the same embodiment. Furthermore, the described specific feature, structure, material or characteristic can be involved in any one or more embodiments in a proper way.

In the embodiments of the present specification, the terminology “or” includes the combination of part of listed components, and the combination of all the listed components. For example, the described “A or B” includes only A, only B, and both A and B. Moreover, the terminologies “a” and “the” before the element or component of the present invention do not limit the number of element or component. Therefore, the terminologies “a” and “the” should be read as including one or at least one. Besides, the singular form of element or component also includes the plural form, unless the number clearly refers to the singular form.

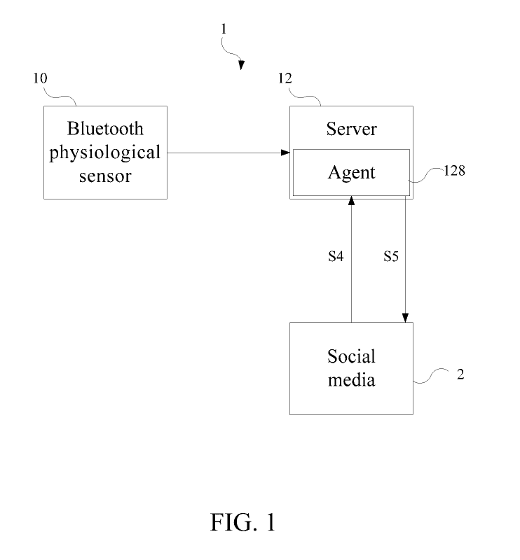

Please refer to FIG.1. FIG. 1 shows a schematic diagram of one embodiment of the interactive physiology monitoring and sharing system 1 of the present invention. The interactive physiology monitoring and sharing system 1 of the present invention is applied to a social media 2. The system comprises a Bluetooth physiological sensor 10 and a server 12. The Bluetooth physiological sensor 10 is used for sensing and sending physiological data. The server 12 is connected to the social media 2 and the Bluetooth physiological sensor 10 for receiving the physiological data, wherein an agent 128 is pre-stored in the server 12. When the server 12 receives a user message S4 from the social media 2, the agent 128 analyzes the user message S4 and the physiological data for generating and sending a feedback message S5 to the social media 2.

In practice, the social media 2 may be LINE, WeChat, Facebook Messenger, Skype, Kik, QQ, WhatsApp, Viber, Telegram, Twitter, Weibo, Tumblr, Snapchat, or any kinds of programs, applications, or platforms to communicate with different terminal devices.

In practice, the Bluetooth physiological sensor 10 may be a weight meter, a wristband, a blood pressure monitor, a blood glucose meter, an oximeter, a thermometer, a fatigue sensor, a respiratory sensor, a cardiac electrical pulse sensor, a heartbeat sensor or any sensors allowing the user to detect immediate physiological conditions.

In practice, Bluetooth technology has the advantages of high popularity, high security encryption, high stability, low construction cost, low interference, low energy consumption and fast connection establishment. Therefore, compared to other wireless technologies, Bluetooth technology is more suitable to serve as a physiological sensing device built-in communication technology.

In an embodiment, the Bluetooth physiological sensor 10 is preset to communicate with the server 12 after sensing the physiological data, by the Bluetooth and the internet (TCP/IP or other protocols) to conduct the communication. For example, a user A measures weight by a Bluetooth weight meter. The weight data not only can be displayed on weight meter interface after measuring, but also can be sent to the server 12 to store by Bluetooth and the internet. Then a user B asks the agent 128 about the weight changing within a week of user A by Facebook Messenger. Next, the server 12 provides a related chart about the weight changing within a week of user A, an analysis of the weight changing trend, and a suggestion of paying attention to user A's diet to Facebook Messenger.

In an embodiment, the agent 128 may perform a semantic analysis on the message sent by the user B and convert into the corresponding input parameter. Then, according to the input parameters, the measurement records of the user A within a week is found out from a database. After that, the measurement records are output to the social media 2 in an appropriate format chart. Finally, the meaning of the measurement records are analyzed and converted into natural language for transmitting to the social media 2.

With such a high ratio of market share of social media 2 like Facebook Messenger, users do not have to download other Apps to communicate with the server 12, so the need of additional Apps downloading is eliminated. In addition, since the physiological data such as weight is automatically stored in the server 12, the user does not need to record the value of each measurement. Furthermore, since the physiological data detected by the Bluetooth physiological sensor 10 can be analyzed by agent 128, a simple and effective primary report can be made without medical professionals. Therefore, the medical resources are saved and the user may quickly learn about his/her own physical condition.

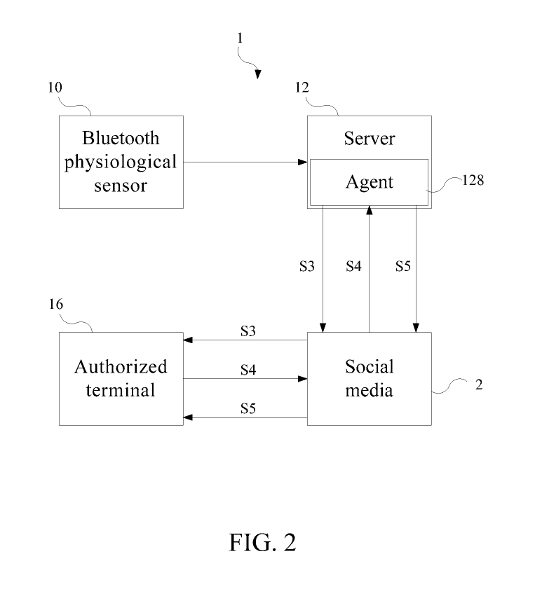

Please refer to FIG. 2. FIG. 2 shows a schematic diagram of another embodiment of the interactive physiology monitoring and sharing system 1 of the present invention. In an embodiment, when the server 12 receives the physiological data in a sensing message, the agent 128 generates and sends a report message S3 containing the physiological data to the social media 2. For example, a user A detects blood pressure by a blood pressure meter. The blood pressure data not only can be displayed on blood pressure meter interface after measuring, but also can be sent to the server 12 to store by Bluetooth and the internet. Then the blood pressure parameters are sent to the social media 2 in an appropriate format chart.

In this embodiment, the server 12 actively sends each measured physiological data as a report message S3, which may help the user A record the physiological data of each measurement, and may also proactively inform a user B about the health status of the user A.

In an embodiment, the interactive physiology monitoring and sharing system 1 further comprises an authorized terminal 16 connected to the social media 2. The authorized terminal 16 is used for sending the user message S4 to the social media 2, and receiving the report message S3 and the feedback message S5 from the social media 2.

In practice, the hardware of the authorized terminal 16 of the present invention may be a smart phone, a personal computer, a tablet or any terminal devices including internet functions, a processing unit and a display interface. However, the hardware must be authorized by the server 12 to receive the report message S3 and the feedback message S5 corresponding to the physiological data. For example, the user A authorizes the user's own smart phone to communicate with the agent 128 through the server 12. When the user A uses the specific Bluetooth physiological sensor 10, only the authorized smart phone will receive the report message S3 and the feedback message S5.

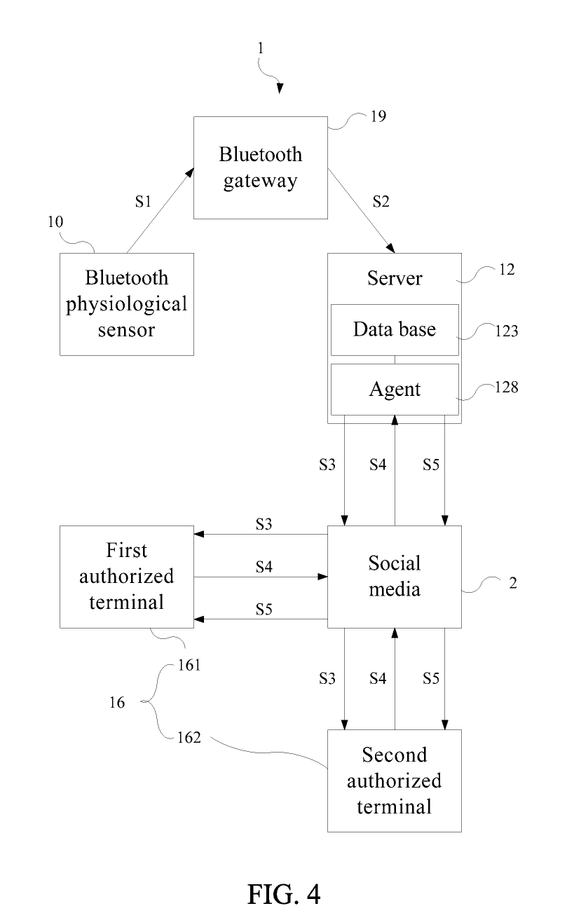

Please refer to FIG.4. FIG. 4 shows a schematic diagram of another embodiment of the interactive physiology monitoring and sharing system 1 of the present invention. In an embodiment, the interactive physiology monitoring and sharing system 1 further comprises a plurality of authorized terminals 16. When the Bluetooth physiological sensor 10 sends the physiological data to the server 12, the server 12 sends the report message S3 to the authorized terminals 16 respectively via the social media 2. In an embodiment, when a first authorized terminal 161 sends the user message S4 to the server 12, the server 12 sends the feedback message S5 to the authorized terminals 16 respectively via the social media 2.

In prior art, the user often establishes an account on social media 2, and the same account may be used in multiple terminal devices. Therefore, in another embodiment, the account of a user A on social media 2 instead of the authorized terminal 16 is authorized by the server 12. If there are multiple hardware devices signed in the account of the user A on social media 2, the multiple hardware devices will all be considered as authorized terminals and receive the report message S3 and the feedback message S5.

In another embodiment, a user A authorizes his smart phone, his tablet and a smart phone of a user B; namely, the above-mentioned devices all be installed with LINE App of the social media 2 to communicate with the server 12. Then, when the user A detects blood glucose by a Bluetooth blood glucose meter, the server 12 sends the report message S3 containing blood glucose data to LINE App of the smart phone of the user A, the tablet of the user A, and the smart phone of the user B through LINE server.

In an embodiment, the authorization method can create a LINE group by a user A's smart phone. This is a dialog that allows multiple accounts. The members of the group include the LINE account of the agent 128 and the user B's LINE account. The user A and B may send the user message S4 by typing in the LINE group, and receive the report message S3 and the feedback message S5 from the agent 128. Thereby, the friends or relatives of the user A and the medical professional may care and receive the user A's healthy status simultaneously.

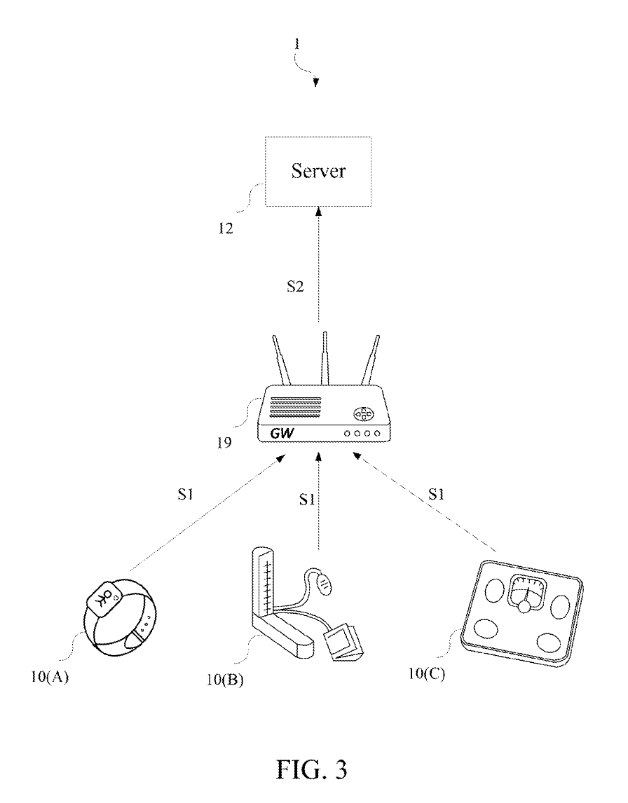

Please refer to FIG.3 and FIG.4. FIG. 3 shows a schematic diagram of one embodiment of the Bluetooth gateway 19 of the present invention. In an embodiment, the interactive physiology monitoring and sharing system 1 further comprises a Bluetooth gateway 19. Wherein the Bluetooth gateway 19 is connected to the Bluetooth physiological sensor 10 through Bluetooth technology for receiving a Bluetooth message S1 containing the physiological data. The Bluetooth gateway 19 is connected to the server 12 via the internet (TCP/IP or other protocols) for sending a sensing message S2 containing the physiological data to the server 12.

In an embodiment, the interactive physiology monitoring and sharing system 1 further comprises a plurality of authorized terminals 16. Wherein each of the authorized terminals 16 is used for sending the user message S4 to the social media 2, and receiving the report message S3 and the feedback message S5 from the social media 2. The Bluetooth message S1 and the sensing message S2 contain a device identifier of the Bluetooth physiological sensor 10 respectively. The device identifier is used for corresponding to a first authorized terminal 161. The agent 128 sends the report message S3 and the feedback message S5 to the first authorized terminal 161 via the social media 2 according to the device identifier.

In an embodiment, since the server 12 may receive the physiological data from multiple authorized terminals 16, the source of each physiological data should be determined. To solve this problem, the Bluetooth message S1 includes a device identifier and the physiological data, and the sensing message S2 also includes the device identifier and the physiological data. Therefore, the server 12 may know which Bluetooth physiological sensor 10 should be corresponded to the physiological data. In this way, it is beneficial for the server 12 to organize, sort and record each the physiological data from the Bluetooth physiological sensor 10. After that, the report message S3 and the feedback message S5 from the corresponding Bluetooth physiological sensor 10 are sent to the authorized terminals 16.

In an embodiment, each of the authorized terminals 16 contains a user identifier, and the device identifier is used for corresponding to a first user identifier. According to the device identifier, the agent 128 sends the report message S3 and the feedback message S5 to at least one authorized terminal 16 containing the first user identifier corresponding to the device identifier.

In practice, with the friend function provided by the LINE App, the authorization between the authorized terminal 16 and the agent 128 can be completed by joining a friend. The agent 128 obtains the user identifier from LINE App. The user identifier may be an account string or a QR code, so the authorized terminal 16 may scan the QR code to complete the authorization. In the authorizing procedure, the agent 128 can obtain the user identifier of the authorized terminal 16 by communicating with the social media 2. By the user identifier, the agent 128 may sends the report message S3 and the feedback message S5 to the authorized terminal 16.

In practice, the user A sets a Bluetooth bracelet 10(A) to correspond to his user identifier of LINE. While the user A exercises with the Bluetooth bracelet 10(A), the detected heartbeat is packaged to a Bluetooth message S1 and sent to the Bluetooth gateway 19. Then the Bluetooth gateway 19 repackages the Bluetooth message S1 to the sensing message S2, and the sensing message S2 is sent to the server 12. After that, the agent 128 determines the corresponding authorized terminal 16 according to the device identifier contained in the sensing message S2. After the agent 128 analyzes the physiological data, the report message S3 about the heartbeat is sent to the authorized terminal 16 bound to the user A through the social media 2.

In practice, the Bluetooth gateway 19 may provide an identifier code of LINE. The identifier code is corresponding to the agent 128 and used for establishing the relationship between the agent 128 and other accounts of LINE. Since a user A and a user B complete the authorization by scanning the identifier code of LINE, the user A has a first authorized terminal 161 and the user B has a second authorized terminal 162. When the Bluetooth gateway 19 receives the Bluetooth message S1, and the sensing message S2 is sent to the server 12, the agent 128 sends the report message S3 to the first authorized terminal 161 and the second authorized terminal 162 by LINE.

In an embodiment, the server 12 further comprises a data base 123. While the server 12 receives the physiological data, the physiological data is stored in the data base 123, and then the agent 128 determines the source and validity of the physiological data. In an embodiment, the agent 128 generates a tag of the physiological data according to the user message S4, and the tag comprises an identity of the subject, data source device and validity of the physiological data to categorize the each physiological data by its characteristics. In practice, the user A wears an ECG pulse sensor. However, the sensor is not installed correctly, so that the measured heart rate is below 20 beats/minute. The server 12 receives and determines the physiological data invalid and further sends a report message S3 to remind the user A that the sensor should be installed again.

In an embodiment, the data base 123 may store the physiological data for a long period of time and classify the physiological data according to the tag. Therefore, the users can look over their long-term physiologic data to evaluate ongoing medications or future medical plans. In an embodiment, with the permission of the user, the database 123 can be used as an important basic data in the medical field for further exploration of medical research or course evaluation.

Since one Bluetooth physiological sensor 10 could be shared by many users, in an embodiment, different users can be identified by a fingerprint recognizer, a face recognizer, or a chosen interface. In another embodiment, the agent 128 may identify the user according to the characteristics of physiological data. For example, a Bluetooth weight meter 10(C) is often used by a user A and a user B. The weight of the user A is 50 kg and the body fat percentage is 12%; the weight of the user B is 80 kg and the body fat percentage is 28%. When the Bluetooth physiological sensor 10 senses a weight of 53 kg and a body fat percentage of 11%, the server 12 presets a tag of the physiological data for user A, and sends a report message S3 to the authorized terminal 16 of the user A.

In another embodiment, the agent 128 receives the user message S4 to determine the physiological data owner. For example, a Bluetooth blood pressure meter 10(B) is bound to a first authorized terminal 161 and other authorized terminals 16. When the agent 128 receives a user message S4 “I'm going to measure my blood pressure.” from the first authorized terminal 161 before the agent 128 receives a physiological data from the Bluetooth blood pressure meter 10(B), the agent 128 determines that the physiological data is corresponding to the first authorized terminal 161, and sends a report message S3 to the first authorized terminal 161.

In contrast, a plurality of the Bluetooth physiological sensors 10 may direct to one user. For example, a user A uses a Bluetooth blood pressure meter 10(B) and a Bluetooth weight meter 10(C) often. Then the physiological data may be labeled as the same user or bound to the same authorized terminal 16. Therefore, the user A may interact with the agent 128 in the authorized terminal 16 to simultaneously obtain the physiological data measured by the Bluetooth blood pressure meter 10(B) and the Bluetooth weight meter 10(C).

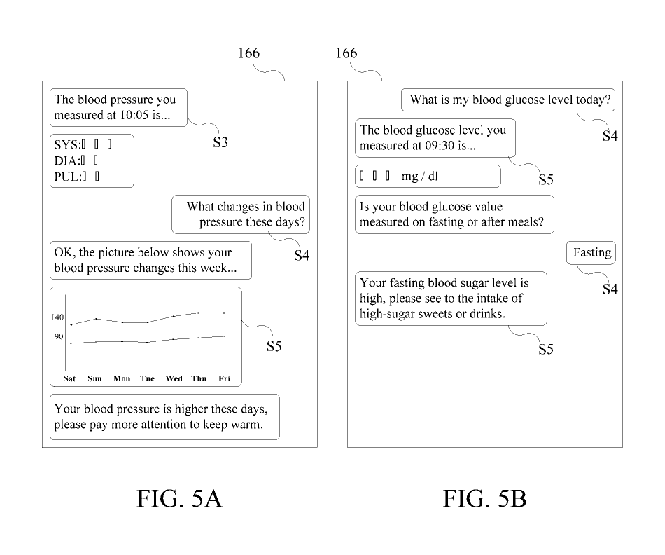

Please refer to FIG. 4, FIG. 5A and FIG. 5B. FIG. 5A and FIG. 5B show a schematic diagram of an embodiment of the interaction between the authorized terminal 16 and the server 12 displayed in the interface 166 of the authorized terminal 16 respectively. In an embodiment, the server 12 displays the report message S3 and the feedback message S5 by the interface 166 of the authorized terminal 16. The user may utilize an input device or the interface 166 to send the user message S4 to the server 12. The interaction function provided by the agent 12 may offer physiological data related information, for example, a value measured on that day, an average value of the past time interval, or a value measured in a specific time period of the past time interval.

In an embodiment, as shown in FIG. 5B, the feedback message S5 may be a question or an advisement. In practice, the agent 128 makes inquiries of the user for obtaining more tags of the physiological data, or makes advisements the user may interest in. For example, according to the blood pressure value of the user and the weather that day, the agent 128 may provide a wearing advice. In another embodiment, the agent 128 may provide a diet advice according to the blood glucose value of the user in a past few days.

In an embodiment, the report message S3 and the feedback message S5 may be a text, an audio stream, a video message, a word, a sentence, a voice, a movie, an animation, an image or a picture. By selecting ways of representation, the agent 128 informs the meaning of the information in the feedback message S5 and the report message S3 simply and clearly.

The Bluetooth in the mentioned embodiment may be any versions of the Bluetooth communication protocol defined by the Bluetooth Special Interest Group, such as the Bluetooth 2.0 protocol series, the Bluetooth 3.0 protocol series, the Bluetooth 4.0 protocol series or the Bluetooth 5.0 protocol series Bluetooth devices.

The internet in the mentioned embodiment may be a wired connection, a wireless LAN or a mobile communication network.

The wired connection in the mentioned embodiment may be Ethernet, FTTx, Power line communications, and networks able to deliver the network packets.

The wireless LAN in the mentioned embodiment may be any versions of the 802.11 protocol defined by Wi-Fi or IEEE, such as 802.11, IEEE 802.11a, 802.11b, 802.11g, 802.11h, 802.11n, 802.11p, 802.11ac, 802.11ah, 802.11aq, 802.11ax and so on.

The mobile communications network in the mentioned embodiment may be LoRa, Sigfox or the network agreement of Release 4, Release 5, Release 6, Release 7, Release 8, Release 9, Release 10, Release 11, Release 12, Release 13, or Release 14 released by 3GPP, or other mobile communications network agreement approved by ITU.

Compare to the prior art, the remote Bluetooth device communication system of the present invention sends the physiological data to the server by Bluetooth technology, so the sensor do not require costly storage and computing ability. Instead of medical professionals, the agent can analyze and explain the physiological data, so the user can get the corresponding report instantly. The social media is used to make interaction between the user and the agent to save another job of installing App. Moreover, the social media can be used to make interaction between a plurality of users and the agent to understand each other's health. Furthermore, the agent may communicate with the user in a natural language due to the chatbot program. Then the physiological data is tagged to identify according to the user message or the physiological characteristic. At last, since the authorized sensors, accounts, and terminals are used to identify the user, the present invention could communicate with the target user.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. An interactive physiology monitoring and sharing system applied to a social media, comprising:
a Bluetooth physiological sensor for sensing and sending physiological data; and
a server connected to the social media and the Bluetooth physiological sensor for receiving the physiological data, wherein an agent is pre-stored in the server;
wherein, when the server receives a user message from the social media, the agent analyzes the user message and the physiological data for generating and sending a feedback message to the social media.

2. The interactive physiology monitoring and sharing system of claim 1, wherein when the server receives the physiological data, the agent generates and sends a report message containing the physiological data to the social media.

3. The interactive physiology monitoring and sharing system of claim 2, further comprising an authorized terminal connected to the social media, wherein the authorized terminal is used for sending the user message to the social media, and receiving the report message and the feedback message from the social media.

4. The interactive physiology monitoring and sharing system of claim 2, further comprising a plurality of authorized terminals, wherein when the Bluetooth physiological sensor sends the physiological data to the server, the server sends the report message to the authorized terminals respectively via the social media.

5. The interactive physiology monitoring and sharing system of claim 4, wherein when a first authorized terminal sends the user message to the server, the server sends the feedback message to the authorized terminals respectively via the social media.

6. The interactive physiology monitoring and sharing system of claim 2, wherein the feedback message and the report message comprises at least one of a text, an audio stream, a video message, an animation, or an image respectively.

7. The interactive physiology monitoring and sharing system of claim 1, further comprising a Bluetooth gateway, wherein the Bluetooth gateway is connected to the Bluetooth physiological sensor through Bluetooth technology for receiving a Bluetooth message containing the physiological data, and the Bluetooth gateway is connected to the server via the Internet for sending a sensing message containing the physiological data to the server.

8. The interactive physiology monitoring and sharing system of claim 7, further comprising a plurality of authorized terminals, wherein each of the authorized terminal is used for sending the user message to the social media and receiving the report message and the feedback message from the social media; the sensing message contain a device identifier of the Bluetooth physiological sensor respectively; the device identifier is used for corresponding to a first authorized terminal, and the agent sends the report message and the feedback message to the first authorized terminal via the social media according to the device identifier.

9. The interactive physiology monitoring and sharing system of claim 8, wherein each of the authorized terminal contains a user identifier, and the device identifier is used for corresponding to a first user identifier; according to the device identifier, the agent sends the report message and the feedback message to at least one authorized terminal containing the first user identifier corresponding to the device identifier.

10. The interactive physiology monitoring and sharing system of claim 1, wherein the agent comprises at least one of a robotic program, a chatbot, an artificial intelligence program, or a machine learning program.

11. The interactive physiology monitoring and sharing system of claim 1, wherein the agent generates a tag of the physiological data according to the user message, and the tag comprises an identity of the subject and validity of the physiological data.
